(19) United States
(12) Patent Application Publication
(10) Pub. No.: US 2015/0250644 A1
(43) Pub. Date: Sep. 10, 2015

(54) Prefilled Poultice Wraps and Systems

(71) Applicant: THE BETTER BANDAGE COMPANY, Mississauga (CA)

(72) Inventors: Chad Eric Blough, Mono (CA); John David Rogers, Amaranth (CA); James Michael Phillips, Mono (CA)

(21) Appl. No.: 14/425,653

(22) PCT Filed: Sep. 4, 2013
(86) PCT No.: PCT/IB2013/001953

§ 371 Date: Mar. 4, 2015

Related U.S. Application Data
(60) Provisional application No. 61/697,092, filed on Sep. 5, 2012.
(60) Provisional application No. 61/764,335, filed on Feb. 13, 2013.

(51) Int. Cl. A61F 7/03; B65B 51/10; A61D 9/00; B65B 11/50

(52) U.S. Cl. CPC A61F 7/03; B65B 11/50; B65B 51/10; A61D 9/00; A61F 2007/0029

(57) ABSTRACT

The present disclosure provides a prefilled wrap, such as a prefilled poultice wrap, for applying to the body of a human or animal. The prefilled wrap includes a matrix defining a plurality of pockets therein. The prefilled wrap also includes a dry ingredient formulation contained in each of the plurality of pockets. The present disclosure also provides a prefilled wrap system for applying to the body of a human or animal. The prefilled wrap system includes an outer casing configured to wrap around a portion of the body of the human or animal. The prefilled wrap system also includes a prefilled wrap removably received in the outer casing. The outer casing is configured to hold the prefilled wrap against the body of the human or animal. Related methods of manufacturing the prefilled wrap, applying the prefilled wrap, and applying the prefilled wrap system are also disclosed.

### FIELD OF THE DISCLOSURE

The present disclosure relates generally to wraps and dressings, and more particularly to prefilled wraps, prefilled wrap systems, prefilled poultice wraps, prefilled poultice wrap systems, and methods of using such wraps and systems for application to the body of a human or animal.

### BACKGROUND OF THE DISCLOSURE

Various types of wraps and dressings are applied to the body of a human or animal for medical or cosmetic purposes. In certain medical applications, a wrap may be applied to a portion of the body to protect a wound, promote healing, prevent infection, or alleviate pain. In certain cosmetic applications, a wrap may be applied to a portion of the body to moisturize, cleanse, soothe, beautify, or otherwise enhance the skin or fur of the human or animal. The wrap may include one or more ingredients that are able to achieve the desired medical or cosmetic effect. Many conventional wraps may present a problem in evenly maintaining the ingredients against the body over time, particularly if the ingredients are applied to the body in a liquid or gel-like state. As a result, the effectiveness of the wrap may be limited. Additionally, in certain applications, the ingredients may be applied directly to the body, and the wrap may be applied over the ingredients. Although this technique may be used for applying different types of ingredients to the body for medical or cosmetic purposes, it is widely recognized as the standard method of applying poultices.

A poultice is often used as a first-aid measure for the body of a human or animal, providing several potential benefits. For example, a poultice may be used in a cooling manner to control or draw out inflammation, reduce swelling and fluid build-up, alleviate pain, or generally cool down muscles after significant use. Alternatively, a poultice may be used in a warming manner to treat muscle tension, loosen stiff joints, ease tendon or ligament injuries, soothe sore feet or hooves, control bruising, or to draw out abscesses. Accordingly, a poultice may serve to address an existing ailment of the body or proactively reduce the likelihood of injury or secondary complications. Use of a poultice is especially beneficial for areas of the body that are subjected to significant stress and strain, such as arms, legs, feet, and joints. Although specific applications vary widely, poultices are most commonly used in the veterinary field on the legs and hooves of horses.

Conventionally, the poultice is a mud-like material that retains moisture for a period of time in order to provide a cooling or warming effect. Specific compositions vary, although common poultice ingredients include dirt, clay, oats, bran, tragacanth, and other natural or synthetic materials that have a high water-holding capacity. As the ingredients dry out while the poultice is being used, they assist in drawing out heat and inflammation from the body and provide a conductive surface to warm or cool the body part depending on the specific need. The poultice may also include certain additives to enhance the cooling or warming effect, or to provide a medicating effect. For example, common poultice additives include menthol, eucalyptus, arnica, peppermint oil, lavender oil, melaleuca oil, camphor, glycerin, clay, Epsom salt and boric acid.

Traditional poultice application involves mixing and hydrating the ingredients, or using premixed preparations, and then applying the mud-like mixture directly on a portion of the body to be treated. Accordingly, preparation and use of the poultice is often a very messy and wasteful process. The poultice is formed as a coating over the portion of the body, which is then covered with several barrier layers, such as brown paper and plastic material, to keep the poultice moist for an extended period of time. The barrier layers are then covered with a conventional cotton padding or outer wrap to hold the poultice against the body. The poultice may remain in contact with the body for 12 to 18 hours or perhaps longer in certain applications. By that point in time, the poultice has dried out significantly, becomes ineffective as a poultice, and often has bonded to the skin, hair, fur, nails, or hooves. Accordingly, the poultice is difficult to remove without thoroughly washing and scrubbing the affected portion of the body with water. Naturally, the poultice is somewhat rehydrated as it is being washed off and creates a mess in the surrounding area which requires considerable clean up and concerns with waste water drains. If the poultice is not removed completely from the body, remaining poultice debris may result in skin irritation and discomfort. Ultimately, traditional poultice application and removal is time-consuming, costly and tedious.

Thus, there is a need for improved devices, systems, and methods for applying ingredients, such as a poultice, to the body of a human or animal. Such devices and systems should be of simple construction that requires minimal preparation and facilitates quick application and removal. Additionally, such devices and systems should eliminate the mess associated with applying and removing the ingredients according to conventional methods. Furthermore, such devices and systems should be configured to hold the ingredients against a desired portion of the body without allowing them to bond to the body.

### SUMMARY OF THE DISCLOSURE

The devices, systems, and methods described herein allow for quick and easy application and clean removal of ingredients used on the body of a human or animal for medical or cosmetic purposes. As described in detail below, such devices, systems, and methods address the abovementioned problems associated with traditional application of such ingredients with wraps or dressings.

In one aspect, the present disclosure includes a prefilled wrap for applying to the body of a human or animal. The prefilled wrap includes a matrix defining a plurality of pockets therein. The prefilled wrap also includes a dry ingredient formulation contained in each of the plurality of pockets. The dry ingredient formulation may be able to achieve one or more medical or cosmetic effects. In some embodiments, the matrix includes a first sheet and a second sheet sealed to one another to define the plurality of pockets between the first sheet and the second sheet. In some embodiments, the first sheet and the second sheet are formed of a flexible material configured to allow the matrix to wrap around and contour to the body of the human or animal. In some embodiments, the first sheet and the second sheet are formed of a fluid-permeable material configured to allow a reconstituting fluid to enter the plurality of pockets to reconstitute the dry ingredient formulation. In some embodiments, the first sheet and the second sheet are configured to retain the reconstituted ingredient formulation in each of the plurality of pockets. In some embodiments, the dry ingredient formulation includes at least one ingredient of the group consisting of guar gum, Epsom salt (magnesium sulfate), clay, hyaluronic acid, emollients, rice starch, bran, soybean protein, camphor, lecithin, antibiotics, liniments, homeopathic preparations, and pharmaceuticals. In some embodiments, the dry ingredient formulation includes guar gum and Epsom salt. In some embodiments, the dry ingredient formulation includes clays, guar gum, bran, and Epsom salt. In some embodiments, the matrix is configured to wrap around and contour to a leg or arm of the human or animal. In some embodiments, the matrix is configured to wrap around and contour to the foot or hoof of the human or animal.

According to one embodiment, the present disclosure provides a prefilled poultice wrap for applying to the body of a human or animal. The prefilled poultice wrap includes a matrix defining a plurality of pockets therein. The prefilled poultice wrap also includes a dry poultice formulation contained in each of the plurality of pockets. In some embodiments, the matrix includes a first sheet and a second sheet sealed to one another to define the plurality of pockets between the first sheet and the second sheet. In some embodiments, the first sheet and the second sheet are formed of a flexible material configured to allow the matrix to wrap around and contour to the body of the human or animal. In some embodiments, the first sheet and the second sheet are formed of a fluid-permeable material configured to allow a reconstituting fluid to enter the plurality of pockets to reconstitute the dry poultice formulation. In some embodiments, the first sheet and the second sheet are configured to retain the reconstituted poultice formulation in each of the plurality of pockets. In some embodiments, the dry poultice formulation includes guar gum and Epsom salt. In some embodiments, the dry poultice formulation includes clay, guar gum, and Epsom salt. In some embodiments, the matrix is configured to wrap around and contour to a leg or arm of the human or animal. In some embodiments, the matrix is configured to wrap around and contour to the foot or hoof of the human or animal.

In another aspect, the present disclosure includes a system of prefilled wraps for applying to the body of a human or animal. The system includes a plurality of prefilled wraps connected in series, and adjacent prefilled wraps are connected to one another along a perforated or non-perforated seam. Each of the plurality of prefilled wraps includes a matrix defining a plurality of pockets therein. Each of the plurality of prefilled wraps also includes a dry ingredient formulation contained in each of the plurality of pockets. In some embodiments, the seam is configured to allow the adjacent prefilled wraps to be separated from one another at the seam without damage to the matrices of the adjacent prefilled wraps. In some embodiments, the adjacent prefilled wraps are gathered around one another in series so that the plurality of prefilled wraps forms a roll. In some embodiments, the adjacent prefilled wraps are folded such that the plurality of prefilled wraps forms a stack.

According to one embodiment, the present disclosure provides a system of prefilled poultice wraps for applying to the body of a human or animal. The system includes a plurality of prefilled poultice wraps connected in series, and adjacent prefilled poultice wraps are connected to one another along a perforated or non-perforated seam. Each of the plurality of prefilled poultice wraps includes a matrix defining a plurality of pockets therein. Each of the plurality of prefilled poultice wraps also includes a dry poultice formulation contained in each of the plurality of pockets. In some embodiments, the seam is configured to allow the adjacent prefilled poultice wraps to be separated from one another at the seam without damage to the matrices of the adjacent prefilled poultice wraps. In some embodiments, the adjacent prefilled poultice wraps are gathered around one another in series so that the plurality of prefilled poultice wraps forms a roll. In some embodiments, the adjacent prefilled poultice wraps are folded such that the plurality of prefilled poultice wraps forms a stack.

In yet another aspect, the present disclosure includes a prefilled wrap system for application to the body of a human or animal. The prefilled wrap system includes a protective outer casing configured to wrap around a portion of the body of the human or animal. The prefilled wrap system also includes a prefilled wrap removably received in the outer casing. The prefilled wrap includes a matrix defining a plurality of pockets therein. The prefilled wrap also includes a dry ingredient formulation contained in each of the plurality of pockets. The outer casing is configured to hold the prefilled wrap against the body of the human or animal. In some embodiments, the outer casing is configured to wrap around and contour to a foot or hoof of the human or animal, and the prefilled wrap is configured to wrap around and contour to the foot or hoof. In some embodiments, the outer casing includes a base portion configured to be positioned under the foot or hoof, a front portion configured to be positioned over the front of the foot or hoof, a back portion configured to be positioned over the back of the foot or hoof, and side portions configured to be positioned over the sides of the foot or hoof. In some embodiments, the outer casing also includes a base inner stabilizing layer positioned on and adhered to an interior surface of the base portion, and a front inner stabilizing layer positioned on and adhered to an interior surface of the front portion. In some embodiments, the prefilled wrap includes a base portion configured to be positioned under the foot or hoof, and a front portion configured to be positioned over the front of the foot or hoof. In some embodiments the base portion of the prefilled wrap is positioned against the base inner stabilizing layer of the outer casing, and the front portion of the prefilled wrap is positioned against the front inner stabilizing layer portion of the outer casing.

According to one embodiment, the present disclosure provides a prefilled poultice wrap system for application to the body of a human or animal. The prefilled poultice wrap system includes a protective outer casing configured to wrap around a portion of the body of the human or animal. The prefilled poultice wrap system also includes a prefilled poultice wrap removably received in the outer casing. The prefilled poultice wrap includes a matrix defining a plurality of pockets therein. The prefilled poultice wrap also includes a dry poultice formulation contained in each of the plurality of pockets. The outer casing is configured to hold the prefilled poultice wrap against the body of the human or animal. In some embodiments, the outer casing is configured to wrap around and contour to a foot or hoof of the human or animal, and the prefilled poultice wrap is configured to wrap around and contour to the foot or hoof. In some embodiments, the outer casing includes a base portion configured to be positioned under the foot or hoof, a front portion configured to be positioned over the front of the foot or hoof, a back portion configured to be positioned over the back of the foot or hoof, and side portions configured to be positioned over the sides of the foot or hoof. In some embodiments, the outer casing also includes a base inner stabilizing layer positioned on and adhered to an interior surface of the base portion, and a front inner stabilizing layer positioned on and adhered to an interior surface of the front portion. In some embodiments, the prefilled poultice wrap includes a base portion configured to be positioned under the foot or hoof, and a front portion configured to be positioned over the front of the foot or hoof. In some embodiments the base portion of the prefilled poultice wrap is positioned against the base inner stabilizing layer of the outer casing, and the front portion of the poultice wrap is positioned against the front inner stabilizing layer portion of the outer casing.

In another aspect, the present disclosure includes a method of applying a prefilled wrap to the body of a human or animal. The method includes the step of providing a prefilled wrap including a matrix defining a plurality of pockets therein. The prefilled wrap also includes at least one dry ingredient formulation contained in each of the plurality of pockets. The method also includes the step of reconstituting the prefilled wrap by applying a reconstituting fluid to the at least one dry ingredient formulation. The method further includes the step of wrapping the reconstituted poultice wrap around a portion of the body of the human or animal.

According to one embodiment, the present disclosure provides a method of applying a prefilled poultice wrap to the body of a human or animal. The method includes the step of providing a prefilled poultice wrap including a matrix defining a plurality of pockets therein. The prefilled poultice wrap also includes at least one dry poultice formulation contained in each of the plurality of pockets. The method also includes the step of reconstituting the prefilled poultice wrap by applying a reconstituting fluid to the at least one dry poultice formulation. The method further includes the step of wrapping the reconstituted poultice wrap around a portion of the body of the human or animal.

In yet another aspect, the present disclosure includes a method of applying a prefilled wrap system to the body of a human or animal. The method includes the step of providing a prefilled wrap system including a protective outer casing and a prefilled wrap received in the outer casing. The prefilled wrap includes a matrix defining a plurality of pockets therein. The prefilled wrap also includes at least one dry ingredient formulation contained in each of the plurality of pockets. The method also includes the step of reconstituting the prefilled wrap by applying a reconstituting fluid to the at least one dry ingredient formulation. The method further includes the step of positioning the prefilled wrap system over a portion of the body of the human or animal, so that the outer casing holds the prefilled wrap against the portion of the body.

According to one embodiment, the present disclosure provides a method of applying a prefilled poultice wrap system to the body of a human or animal. The method includes the step of providing a prefilled poultice wrap system including a protective outer casing and a prefilled poultice wrap received in the outer casing. The prefilled poultice wrap includes a matrix defining a plurality of pockets therein. The prefilled poultice wrap also includes at least one dry poultice formulation contained in each of the plurality of pockets. The method also includes the step of reconstituting the prefilled poultice wrap by applying a reconstituting fluid to the at least one dry poultice formulation. The method further includes the step of positioning the prefilled poultice wrap system over a portion of the body of the human or animal, so that the outer casing holds the prefilled poultice wrap against the portion of the body.

In still another aspect, the present disclosure includes a method of manufacturing a prefilled wrap to apply to the body of a human or animal. The method includes the step of providing a first sheet, a second sheet, and at least one dry ingredient formulation. The method also includes the step of depositing the at least one dry ingredient formulation between the first sheet and the second sheet. The method further includes the step of sealing the first sheet to the second sheet to form a prefilled wrap including a matrix defining a plurality of pockets between the first sheet and the second sheet, so that the at least one dry ingredient formulation is contained in each of the plurality of pockets.

According to one embodiment, the present disclosure provides a method of manufacturing a prefilled poultice wrap to apply to the body of a human or animal. The method includes the step of providing a first sheet, a second sheet, and at least one dry poultice formulation. The method also includes the step of depositing the at least one dry poultice formulation between the first sheet and the second sheet. The method further includes the step of sealing the first sheet to the second sheet to form a prefilled poultice wrap including a matrix defining a plurality of pockets between the first sheet and the second sheet, so that the at least one dry poultice formulation is contained in each of the plurality of pockets.

These and other aspects and embodiments of the present disclosure will become apparent to one of ordinary skill in the art upon review of the following detailed description when taken in conjunction with the several drawings and the appended claims.

### BRIEF DESCRIPTION OF THE DRAWINGS

The detailed description is set forth with reference to the accompanying drawings, which are not necessarily drawn to scale. The use of the same reference numerals indicates similar or identical items. Certain embodiments may utilize elements, components, and/or configurations other than those illustrated in the drawings, and some elements, components, and/or configurations may not be present in certain embodiments. Throughout this disclosure, depending on the context, singular and plural terminology may be used interchangeably.

FIG. 1 illustrates a top view of a prefilled poultice wrap according to one embodiment of the present disclosure.

FIG. 2 illustrates a partial section view of the prefilled poultice wrap of FIG. 1.

FIG. 3 illustrates a top view of a prefilled poultice wrap according to another embodiment of the present disclosure.

FIG. 4 illustrates a partial section view of the prefilled poultice wrap of FIG. 3.

FIG. 5 illustrates an upper perspective view of a protective outer casing of a prefilled poultice wrap system according to one embodiment of the present disclosure.

FIG. 6 illustrates a top view of a base portion, a back portion, side portions, and a flexible tether of the protective outer casing of FIG. 5.

FIG. 7 illustrates a bottom view of a base portion, a back portion, side portions, a flexible tether, an adhesive strip, and an adhesive tail piece of the protective outer casing of FIG. 5.

FIG. 8 illustrates a top view of a front portion of the protective outer casing of FIG. 5.

FIG. 9 illustrates a top view of an adhesive tail piece of the protective outer casing of FIG. 5.

FIG. 10 illustrates a top view of an adhesive strip of the protective outer casing of FIG. 5.

FIG. 11 illustrates a top view of an adhesive strip of the protective outer casing of FIG. 5.

FIG. 12 illustrates a side view of a front stabilizing layer of the protective outer casing of FIG. 5.

FIG. 13 illustrates a top view of the front stabilizing layer of FIG. 12.

FIG. 14 illustrates a top view of a base stabilizing layer of the protective outer casing of FIG. 5.

FIG. 15 illustrates a front view of the base stabilizing layer of FIG. 14.

FIG. 16 illustrates an upper perspective view of a protective outer casing of a prefilled poultice wrap system according to another embodiment of the present disclosure.

FIG. 17 illustrates a top view of a base portion, a back portion, side portions, a flexible tether, a back tie, a tail piece, a debris shield, and a bottom reinforcement insert of the protective outer casing of FIG. 16.

FIG. 18 illustrates a top view of a debris shield of the protective outer casing of FIG. 16.

FIG. 19 illustrates a front view of the debris shield of FIG. 18.

FIG. 20 illustrates a top view of a tail piece of the protective outer casing of FIG. 16.

FIG. 21 illustrates a top view of a back tie of the protective outer casing of FIG. 16.

FIG. 22 illustrates a front view of the back tie of FIG. 21.

### DETAILED DESCRIPTION

The present disclosure may be understood more readily by reference to the following detailed description of specific embodiments. However, it is to be understood that the claimed devices, systems, and methods are not limited to the specific embodiments described herein. It is also to be understood that the terminology used herein is for the purpose of describing the specific embodiments only and is not intended to be limiting.

In one aspect, the present disclosure includes a prefilled wrap for applying to the body of a human or animal. The prefilled wrap includes a matrix defining a plurality of pockets therein. The prefilled wrap also includes a dry ingredient formulation, such as a dry poultice formulation, contained in each of the plurality of pockets. The dry ingredient formulation may be able to achieve one or more medical or cosmetic effects. In some embodiments, the dry ingredient formulation includes at least one ingredient of the group consisting of guar gum, Epsom salt (magnesium sulfate), clay, hyaluronic acid, emollients, rice starch, bran, soybean protein, camphor, lecithin, antibiotics, liniments, homeopathic preparations, and pharmaceuticals. The ingredients of the dry ingredient formulation may be provided in a powdered form and may be blended together to achieve a substantially uniform distribution within the pockets. Generally, the prefilled wrap may be manufactured and stored with the matrix and the dry ingredient formulation in a dry state, although the prefilled wrap may be reconstituted for use by applying a reconstituting fluid thereto. Preferably, the matrix is configured to substantially retain the reconstituted ingredient formulation in each of the pockets. Accordingly, when the prefilled wrap is in use, the matrix may provide a barrier between the reconstituted ingredient formulation and the body of the human or animal, and further may eliminate the mess associated with traditional poultice application and removal. In some embodiments, the matrix provides a semi-permeable barrier between the reconstituted ingredient formulation and the body, allowing a portion of the reconstituted ingredient formulation to slowly migrate therethrough and come into contact with the body. The semi-permeable barrier also may allow for the reconstituting fluid to slowly evaporate from the reconstituted ingredient formulation during use of the prefilled wrap.

In some embodiments, the dry ingredient formulation may be configured to expand when the prefilled wrap is reconstituted for use by applying the reconstituting fluid thereto. Specifically, one or more ingredients of the dry ingredient formulation may be configured to absorb the reconstituting fluid, resulting in expansion of the ingredient formulation. In one embodiment, the dry ingredient formulation completely fills each of the pockets when the prefilled wrap is in the dry state. During reconstitution of the prefilled wrap, the ingredient formulation may expand such that the pockets swell and retain the reconstituted ingredient formulation therein under tension. Accordingly, the swollen pockets may maintain the reconstituted ingredient formulation adjacent the body of the human or animal when the prefilled wrap is applied thereto. The swollen pockets also help the wrap maintain its rigidity and form against the body. In this manner, the prefilled wrap may enhance the medical or cosmetic effects achieved by maximizing the amount of the reconstituted ingredient formulation that slowly migrates through the matrix and comes into contact with the body. In another embodiment, the dry ingredient formulation substantially fills each of the pockets when the prefilled wrap is in the dry state, so that the reconstituted ingredient formulation completely fills each of the pockets when the prefilled wrap is in the reconstituted state. During reconstitution of the prefilled wrap, the ingredient formulation may expand such that the pockets swell and retain the reconstituted ingredient formulation therein under tension. Accordingly, the swollen pockets may maintain the reconstituted ingredient formulation adjacent the body of the human or animal when the prefilled wrap is applied thereto. In this manner, the prefilled wrap may enhance the medical or cosmetic effects achieved by maximizing the amount of the reconstituted ingredient formulation that slowly migrates through the matrix and comes into contact with the body. In still another embodiment, the dry ingredient formulation partially fills each of the pockets when the prefilled wrap is in the dry state, so that the reconstituted ingredient formulation completely fills each of the pockets when the prefilled wrap is in the reconstituted state. During reconstitution of the prefilled wrap, the ingredient formulation may expand such that the pockets swell and retain the reconstituted ingredient formulation therein under tension. Accordingly, the swollen pockets may maintain the reconstituted ingredient formulation adjacent the body of the human or animal when the prefilled wrap is applied thereto. In this manner, the prefilled wrap may enhance the medical or cosmetic effects achieved by maximizing the amount of the reconstituted ingredient formulation that slowly migrates through the matrix and comes into contact with the body. According to various embodiments, the size of the pockets and the amount of dry ingredient formulation contained therein may be selected depending on the expansion capability of the dry ingredient formulation, such that the pockets retain the reconstituted ingredient formulation therein under tension without bursting.

In some embodiments, the matrix includes a first sheet and a second sheet sealed to one another to define the plurality of pockets between the first sheet and the second sheet. In some embodiments, the first sheet and the second sheet are formed of a flexible material configured to allow the matrix to wrap around and contour to the body of the human or animal. In some embodiments, the first sheet and the second sheet are formed of a fluid-permeable material configured to allow a reconstituting fluid to enter the plurality of pockets to reconstitute the dry ingredient formulation. For example, the dry ingredient formulation may be reconstituted with water or another fluid. In some embodiments, the first sheet and the second sheet are configured to substantially retain the reconstituted ingredient formulation in each of the plurality of pockets. In some embodiments, the matrix is configured to wrap around and contour to a leg or arm of the human or animal. In some embodiments, the matrix is configured to wrap around and contour to the foot or hoof of the human or animal.

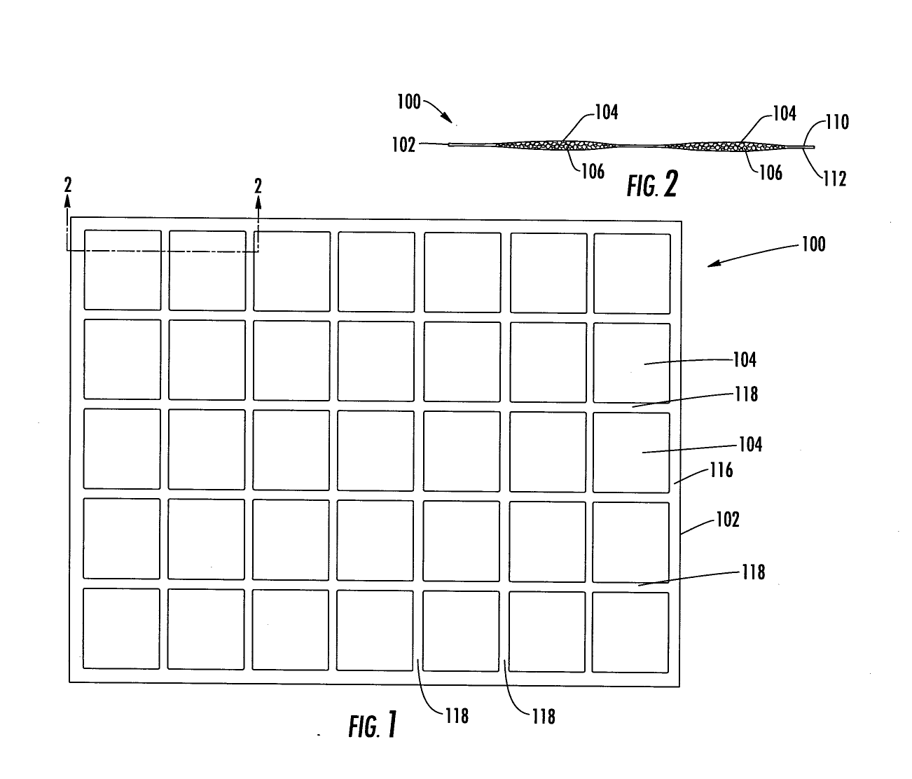

Referring now to the drawings, in which like reference numerals refer to like elements throughout the several views, FIGS. 1 and 2 depict a prefilled poultice wrap 100 according to one embodiment of the disclosure. The prefilled poultice wrap 100 includes a matrix 102 defining a plurality of pockets 104 therein. The prefilled poultice wrap 100 also includes a dry poultice formulation 106 contained in each of the plurality of pockets 104. Generally, the prefilled poultice wrap 100 may be manufactured and stored with the matrix 102 and the dry poultice formulation 106 in a dry state, although the prefilled poultice wrap 100 may be reconstituted for use by applying a reconstituting fluid thereto. Preferably, the matrix 102 is configured to substantially retain the reconstituted poultice formulation 106 in each of the pockets 104. Accordingly, when the prefilled poultice wrap 100 is in use, the matrix 102 may provide a barrier between the reconstituted poultice formulation 106 and the body of the human or animal, and further may eliminate the mess associated with traditional poultice application and removal. In some embodiments, the matrix 102 provides a semi-permeable barrier between the reconstituted poultice formulation 106 and the body, allowing a portion of the reconstituted poultice formulation 106 to slowly migrate therethrough and come into contact with the body. The semi-permeable barrier also may allow for the reconstituting fluid to slowly evaporate from the reconstituted poultice formulation 106 during use of the prefilled poultice wrap 100.

In some embodiments, the dry poultice formulation 106 may be configured to expand when the prefilled poultice wrap 100 is reconstituted for use by applying the reconstituting fluid thereto. Specifically, one or more ingredients of the dry poultice formulation 106 may be configured to absorb the reconstituting fluid, resulting in expansion of the poultice formulation 106. In one embodiment, the dry poultice formulation 106 completely fills each of the pockets 104 when the prefilled poultice wrap 100 is in the dry state. During reconstitution of the prefilled poultice wrap 100, the poultice formulation 106 may expand such that the pockets 104 swell and retain the reconstituted poultice formulation 106 therein under tension. Accordingly, the swollen pockets 104 may maintain the reconstituted poultice formulation 106 against the body of the human or animal when the prefilled poultice wrap 100 is applied thereto. In this manner, the prefilled poultice wrap 100 may enhance the medical or cosmetic effects achieved by maximizing the amount of the reconstituted poultice formulation 106 that slowly migrates through the matrix 102 and comes into contact with the body. In another embodiment, the dry poultice formulation 106 substantially fills each of the pockets 104 when the prefilled poultice wrap 100 is in the dry state, so that the reconstituted poultice formulation 106 completely fills each of the pockets 104 when the prefilled poultice wrap 100 is in the reconstituted state. During reconstitution of the prefilled poultice wrap 100, the poultice formulation 106 may expand such that the pockets 104 swell and retain the reconstituted poultice formulation 106 therein under tension. Accordingly, the swollen pockets 104 may maintain the reconstituted poultice formulation 106 against the body of the human or animal when the prefilled poultice wrap 100 is applied thereto. In this manner, the prefilled poultice wrap 100 may enhance the medical or cosmetic effects achieved by maximizing the amount of the reconstituted poultice formulation 106 that slowly migrates through the matrix 102 and comes into contact with the body. In still another embodiment, the dry poultice formulation 106 partially fills each of the pockets 104 when the prefilled poultice wrap 100 is in the dry state, so that the reconstituted poultice formulation 106 completely fills each of the pockets 104 when the prefilled poultice wrap 100 is in the reconstituted state. During reconstitution of the prefilled poultice wrap 100, the poultice formulation 106 may expand such that the pockets 104 swell and retain the reconstituted poultice formulation 106 therein under tension. Accordingly, the swollen pockets 104 may maintain the reconstituted poultice formulation 106 against the body of the human or animal when the prefilled poultice wrap 100 is applied thereto. In this manner, the prefilled poultice wrap 100 may enhance the medical or cosmetic effects achieved by maximizing the amount of the reconstituted poultice formulation 106 that slowly migrates through the matrix 102 and comes into contact with the body. According to various embodiments, the size of the pockets 104 and the amount of dry poultice formulation 106 contained therein may be selected depending on the expansion capability of the dry poultice formulation 106, such that the pockets 104 retain the reconstituted poultice formulation 106 therein under tension without bursting.

In some embodiments, the matrix 102 includes a first sheet 110 and a second sheet 112 sealed to one another to define the plurality of pockets 104 between the first sheet 110 and the second sheet 112. In some embodiments, the first sheet 110 and the second sheet 112 define only one pocket 104 instead of a plurality of pockets 104. In some embodiments, the first sheet 110 and the second sheet 112 are sealed to one another along a plurality of seams. For example, the first sheet 110 and the second sheet 112 may be sealed to one another by a heat, pressure, radio frequency, stitching, glue, welding, ultra high frequency welding, pressure sensitive adhesives, mechanical fastening or a combination of methods to form a seal along the plurality of seams. In one embodiment, the first sheet 110 and the second sheet 112 are sealed to one another along an outer seam 116 extending about a perimeter of the matrix 102. In one embodiment, the first sheet 110 and the second sheet 112 also are sealed to one another along intermediate seams 118 each extending from one edge of the matrix 102 to an opposite edge of the matrix 102. Some of the intermediate seams 118 may be oriented in a parallel manner, and some of the intermediate seams 118 may be oriented in an intersecting or overlapping manner. Accordingly, the outer seam 116 and the intermediate seams 118 may define the boundaries of each of the plurality of pockets 104. In certain embodiments, the matrix comprises a plurality (e.g., 2 to 10) of pockets in a row, and a plurality (e.g., 2 to 10) of pockets in each column, such that a two dimensional array of separate pockets containing poultice is distributed evenly across the matrix. In some embodiments, each of the pockets 104 is completely separated from the other pockets 104 by one or more of the outer seam 116 and the intermediate seams 118. Accordingly, the pockets 104 are not in communication with one another. In some embodiments, each of the plurality of pockets 104 are completely sealed by the first sheet 110, the second sheet 112, and one or more of the outer seam 116 and the intermediate seams 118. Accordingly, the pockets 104 completely contain the dry poultice formulation 106 and do not include an opening.

In some embodiments, the first sheet 110 and the second sheet 112 are formed of a flexible material configured to allow the matrix 102 to wrap around and contour to the body of the human or animal. Accordingly, the prefilled poultice wrap 100 may be used on various portions of the body, including portions that are significantly contoured or irregularly shaped. After the prefilled poultice wrap 100 is reconstituted for use, the matrix 102 may be particularly amenable to contouring to the body and holding the reconstituted poultice formulation 106 against the body. Preferably, the first sheet 110 and the second sheet 112 are formed of a fluid-permeable material configured to allow a reconstituting fluid to enter the plurality of pockets 104 to reconstitute the dry poultice formulation 106. For example, the dry poultice formulation 106 may be reconstituted with water or another fluid. In some embodiments, the first sheet 110 and the second sheet 112 are configured to substantially retain the reconstituted poultice formulation 106 in each of the plurality of pockets 104. Accordingly, the reconstituted poultice formulation 106 is held against the body and does not substantially exit the matrix 102. Accordingly, when the prefilled poultice wrap 100 is in use, the first sheet 110 and the second sheet 112 may provide a barrier between the reconstituted poultice formulation 106 and the body of the human or animal, and further may eliminate the mess associated with traditional poultice application and removal. In some embodiments, the first sheet 110 and the second sheet 112 provides a semi-permeable barrier between the reconstituted poultice formulation 106 and the body, allowing a portion of the reconstituted poultice formulation 106 to slowly migrate therethrough and come into contact with the body. The semi-permeable barrier also may allow for the reconstituting fluid to slowly evaporate from the reconstituted poultice formulation 106 during use of the prefilled poultice wrap 100.

In some embodiments, the first sheet 110 and the second sheet 112 are formed of a filter paper (fiber paper matrix). Non-limiting examples of suitable filter papers include air filter paper, coffee filter paper, laboratory filter paper, and other types of filter paper that are highly fluid-permeable and that have small pores so that the reconstituted poultice formulation 106 substantially remains contained within the plurality of pockets 104. The filter paper may be relatively lightweight and flexible. Additionally, the filter paper may be relatively durable and have a sufficient dry tensile strength in order to prevent tearing or rupture of the matrix 102, and particularly the pockets 104, during handling and use of the prefilled poultice wrap 100. The filter paper also may have a sufficient wet tensile strength in order to prevent tearing or rupture of the matrix 102 after reconstituting the prefilled poultice wrap 100. Further, the filter paper may have a sufficient air permeability rate in order to allow for adequate air circulation through the matrix 102, particularly when the prefilled poultice wrap 100 is in use. The filler paper can be made of natural or synthetic materials.

In some embodiments, the dry poultice formulation 106 includes guar gum and Epsom salt (magnesium sulfate). Guar gum serves as a thickener or emulsifier that binds the reconstituted poultice formulation 106. Additionally, guar gum is a natural astringent that provides a drying and cooling effect when the prefilled poultice wrap 100 is used on the body. Guar gum provides certain advantages over other natural and synthetic gums that might otherwise be used for a poultice. For example, compared to xanthum gum, guar gum performs better and is able to provide an improved warming effect in certain applications. Compared to locust bean gum, guar gum provides a much better stabilizer and is much more soluble in water. Compared to tapioca, guar gum has about eight times the thickening ability and thus provides a more stable poultice for maintaining contact with the body. Additionally, compared to tragacanth, guar gum serves as a more powerful astringent for drying and cooling the body. Further, compared to carboxymethylcellulose (CMC), a significantly lower quantity (about one fifth) of guar gum is able to achieve a comparable poultice substrate. Guar gum also provides a much higher wettability (ability to absorb a reconstituting fluid, such as water) than other natural and synthetic gums when reconstituted with cold water. Accordingly, the dry poultice formulation 106 including guar gum allows the prefilled poultice wrap 100 to provide both a cold pack effect (i.e., cold therapy for acute injury and inflammation) and a poultice effect in a single application. Notably, due to their low wettability in cold water, other natural and synthetic gums are not able to provide this type of dual-action effect. In some embodiments in which the dry poultice formulation 106 includes guar gum, the prefilled poultice wrap 100 may be configured to provide both a cold pack effect lasting about 1 hour, followed by a poultice effect lasting about 18 hours. Overall, guar gum provides unique and substantial advantages over other known gums and similar ingredients for a poultice. In some embodiments including guar gum and Epsom salt, the Epsom salt comprises a majority of the dry poultice formulation 106 by weight.

In some embodiments, the dry poultice formulation 106 includes clay, guar gum, bran, and Epsom salt. In some embodiments including clay, guar gum, and Epsom salt, the clay comprises a majority of the dry poultice formulation 106 by weight. In some embodiments, the clay includes kaolin clay and bentonite clay. As mentioned above, clay is a common poultice ingredient because of its ability to hold moisture over an extended period of time. However, the prefilled poultice wrap 100 provides certain advantages over many conventional poultices because the dry poultice formulation 106 is manufactured and maintained in a dry state until use, whereas such conventional poultices are hydrated upon manufacture and stored in containers that retain moisture until use. Accordingly, the prefilled poultice wrap 100 is lighter and easier to transport compared to a hydrated poultice. Additionally, the dry nature of the poultice formulation 106 of the prefilled poultice wrap 100 results in a longer shelf life compared to a hydrated poultice. In some embodiments, the dry poultice formulation 106 includes one or more ingredients from the group consisting of antibiotics, liniments, homeopathic preparations, and pharmaceuticals. Such ingredients may be included in the dry poultice formulation 106 without substantially altering the structure of prefilled poultice wrap 100 or the methods of manufacturing or applying the prefilled poultice wrap 100.

In some embodiments, the prefilled poultice wrap 100 is configured to wrap around and contour to a leg or arm of a human or animal. For example, the prefilled poultice wrap 100 may have an elongated shape, such as a rectangle or oval, which is amenable to being wrapped around a generally cylindrical portion of the body. Although certain irregular shapes may be used so that the prefilled poultice wrap 100 may best fit a certain portion of the body, regular shapes may provide the greatest versatility of use. Additionally, the pockets 104 may have certain irregular or regular shapes. Furthermore, the prefilled poultice wrap 100 and the pockets 104 may be formed in many different sizes for optimal use in various human and animal applications.

In some embodiments, each of the plurality of pockets 104 includes an opening, and the dry poultice formulation 106 is contained in a plurality of individual pouches received in the plurality of pockets 104. In such embodiments, each pouch is configured to be inserted in a pocket 104 through the opening. Accordingly, the pouches containing the dry poultice formulation 106 may be inserted in or removed from the pockets 104 of the matrix 102 at any time prior to use of the prefilled poultice wrap 100. Additionally, the configuration of the pouches and openings allow for different dry poultice formulations 106, or other dry ingredient formulations, to be selected and used in combination in the prefilled poultice wrap 100 in a modular manner. Accordingly, the prefilled poultice wrap 100 may be customized easily with a certain combination of dry poultice formulations 106 and other dry ingredient formulations each contained within individual pouches. The openings of the pockets 104 may be formed in the first sheet 110 or the second sheet 112. In some embodiments, the openings are formed along one of the outer seam 116 or the intermediate seams 118. In some embodiments, the pouches are formed of a fluid-permeable material configured to allow a reconstituting fluid to enter the pouches to reconstitute the dry poultice formulations 106 or other dry ingredient formulations. In some embodiments, the pouches are formed of a filter paper similar to that of the first sheet 110 and the second sheet 112. In some embodiments, each of the pouches is completely sealed and does not include an opening.

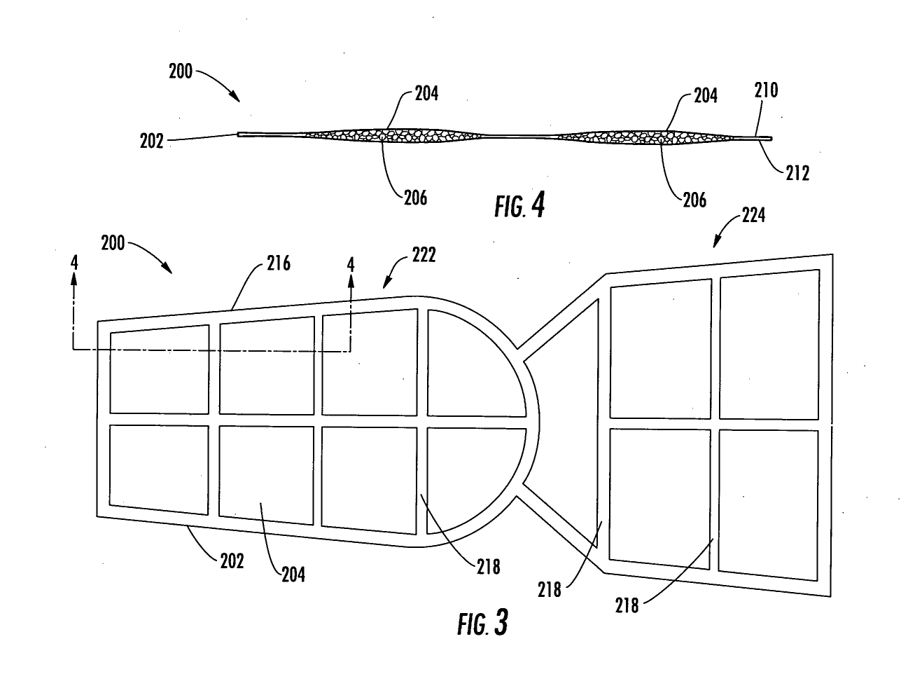

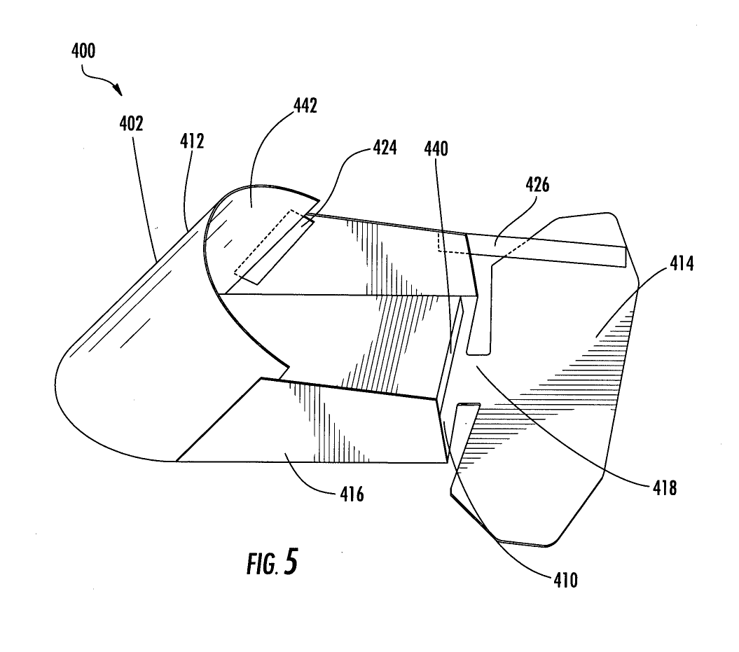

FIGS. 3 and 4 depict a prefilled poultice wrap 200 according to another embodiment of the disclosure. The prefilled poultice wrap 200 similarly includes a matrix 202 defining a plurality of pockets 204 therein. The prefilled poultice wrap 200 also includes a dry poultice formulation 206 contained in each of the plurality of pockets 204. In some embodiments, the matrix 202 includes a first sheet 210 and a second sheet 212 sealed to one another to define the plurality of pockets 204 between the first sheet 210 and the second sheet 212. In one embodiment, the first sheet 210 and the second sheet 212 are sealed to one another along an outer seam 216 extending about a perimeter of the matrix, and also are sealed to one another along intermediate seams 218 each extending from one edge of the matrix 202 toward an opposite edge of the matrix 202. The composition and materials of the matrix 202, the pockets 204, the dry poultice formulation 206, the first sheet 210, the second sheet 212, the outer seam 216, and the intermediate seams 218 of the prefilled poultice wrap 200 may be as described above with respect to the prefilled poultice wrap 100.

In some embodiments, the prefilled poultice wrap 200 is configured to wrap around and contour to a foot or hoof of a human or animal. For example, the prefilled poultice wrap 200 may have an irregular elongated shape, substantially as shown in FIG. 4. Additionally, the pockets 204 may have certain irregular or regular shapes. In some embodiments, the prefilled poultice wrap 200 includes a base portion 222 and a front portion 224. The base portion 222 may be configured to be positioned under the foot or hoof, and the front portion 224 may be configured to be positioned over the front of the foot or hoof. In this manner, the prefilled poultice wrap 200 may be configured to be positioned over and cover the entire foot or hoof, including the coronary band of a horse hoof. In some embodiments, the base portion 222 is shaped to substantially match the profile of the bottom of the foot or hoof. Accordingly, the prefilled poultice wrap 200 may provide a specialized device for applying a poultice, as the prefilled poultice wrap 200 may be shaped to target critical areas of the foot or hoof. Furthermore, the prefilled poultice wrap 200 and the pockets 204 may be formed in many different sizes for optimal use in various human and animal applications where the shapes and sizes of feet and hooves may vary widely.

In another aspect, the present disclosure includes a system of prefilled wraps for applying to the body of a human or animal. For certain applications, prefilled wraps may be manufactured and packaged individually. Alternatively, for other applications, multiple prefilled wraps may be manufactured and packaged together. In one embodiment, the system of prefilled wraps includes a plurality of prefilled wraps connected in series, and adjacent prefilled wraps are connected to one another along a perforated or non-perforated seam. Each of the plurality of prefilled wraps includes a matrix defining a plurality of pockets therein. Each of the plurality of prefilled wraps also includes a dry ingredient formulation contained in each of the plurality of pockets. In some embodiments, the seam is configured to allow the adjacent prefilled wraps to be separated from one another at the seam without damage to the matrices of the adjacent prefilled wraps. In some embodiments, the adjacent prefilled wraps are gathered around one another in series so that the plurality of prefilled wraps forms a roll. In some embodiments, the adjacent prefilled wraps are folded such that the plurality of prefilled wraps forms a stack.

According to one embodiment of the disclosure, a system of prefilled poultice wraps for applying to the body of a human or animal is provided. The system includes a plurality of prefilled poultice wraps 100 connected in series, and adjacent prefilled poultice wraps 100 are connected to one another along a perforated or non-perforated seam. Each of the plurality of prefilled poultice wraps 100 includes a matrix 102 defining a plurality of pockets 104 therein. Each of the plurality of prefilled poultice wraps 100 also includes a dry poultice formulation 106 contained in each of the plurality of pockets 104. In some embodiments, the seam is configured to allow the adjacent prefilled poultice wraps 100 to be separated from one another at the seam without damage to the matrices 102 of the adjacent prefilled poultice wraps 100. In some embodiments, the adjacent prefilled poultice wraps 100 are wrapped around one another in series so that the plurality of prefilled poultice wraps 100 forms a roll. In some embodiments, the adjacent prefilled poultice wraps 100 are folded over one another in series at the seam such that the plurality of prefilled poultice wraps 100 forms a stack. Accordingly, the system provides multiple prefilled poultice wraps 100 in a convenient form for multiple treatments.

In another aspect, the present disclosure includes a prefilled wrap system for applying to the body of a human or animal. For certain applications, a prefilled wrap may be used along with conventional wraps or dressings placed over the prefilled wrap to securely hold it against the body. Alternatively, for other applications, a prefilled wrap may be combined with a case to facilitate placement of the prefilled wrap. In one embodiment, the prefilled wrap system includes a protective outer casing configured to wrap around a portion of the body of the human or animal. The prefilled wrap system also includes a prefilled wrap removably received in the outer casing. The prefilled wrap includes a matrix defining a plurality of pockets therein. The prefilled wrap also includes a dry ingredient formulation contained in each of the plurality of pockets. The outer casing is configured to hold the prefilled wrap against the body of the human or animal. In some embodiments, the outer casing is configured to wrap around and contour to a foot or hoof of the human or animal, and the prefilled wrap is configured to wrap around and contour to the foot or hoof. In some embodiments, the outer casing includes a base portion configured to be positioned under the foot or hoof, a front portion configured to be positioned over the front of the foot or hoof, a back portion configured to be positioned over the back of the foot or hoof, and side portions configured to be positioned over the sides of the foot or hoof. In some embodiments, the outer casing also includes a base inner stabilizing layer positioned on and adhered to an interior surface of the base portion, and a front inner stabilizing layer positioned on and adhered to an interior surface of the front portion. In some embodiments, the prefilled wrap includes a base portion configured to be positioned under the foot or hoof, and a front portion configured to be positioned over the front of the foot or hoof. In some embodiments the base portion of the prefilled wrap is positioned against the base inner stabilizing layer of the outer casing, and the front portion of the prefilled wrap is positioned against the front inner stabilizing layer portion of the outer casing.

According to one embodiment of the disclosure, a prefilled poultice wrap system 300 for applying to the body of a human or animal is provided. The prefilled poultice wrap system 300 includes a case 400 configured to wrap around a portion of the body of the human or animal. The prefilled poultice wrap system 300 also includes a prefilled poultice wrap 200 removably received in the case 400. The prefilled poultice wrap 200 includes a matrix 202 defining a plurality of pockets 204 therein. The prefilled poultice wrap 200 also includes a dry poultice formulation 206 contained in each of the plurality of pockets 204. The case 400 is configured to hold the prefilled poultice wrap 200 against the body of the human or animal. In some embodiments, the case 400 is an outer casing 402 configured to wrap around and contour to a foot or hoof of the human or animal, and the prefilled poultice wrap 200 is configured to wrap around and contour to the foot or hoof. In other embodiments, the case 400 may be another type of covering device configured to wrap around and contour to a different portion of the human or animal, such as an arm, leg, elbow, or knee. In some embodiments, the entire prefilled poultice wrap system 300 is disposable. In other embodiments, the prefilled poultice wrap 200 is disposable and the outer casing 402 is reusable.

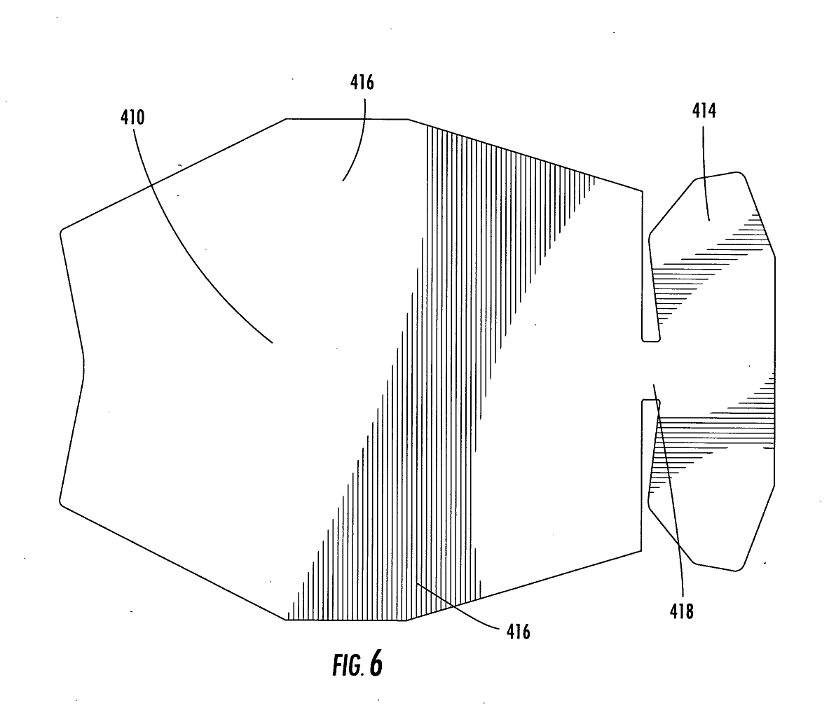

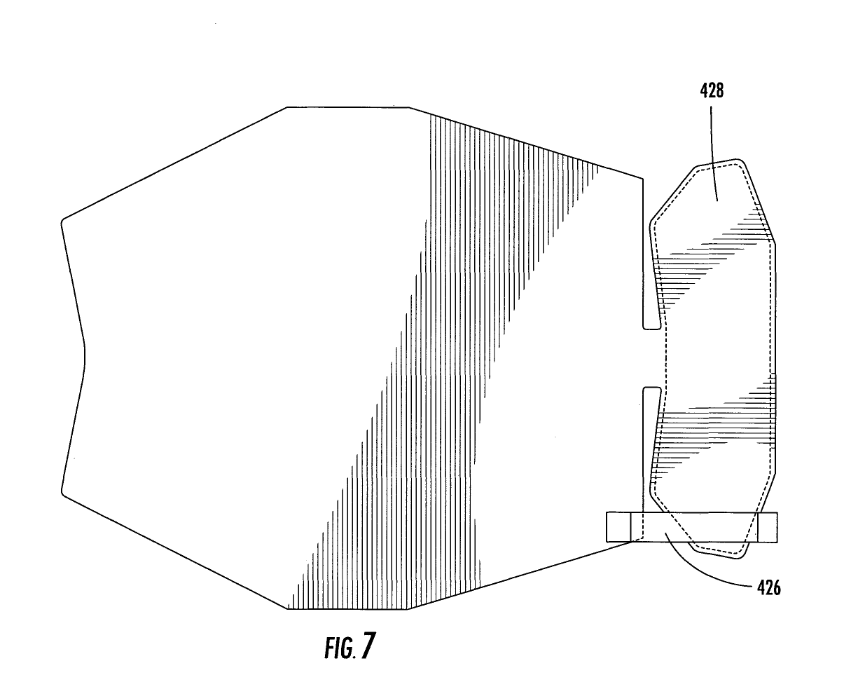

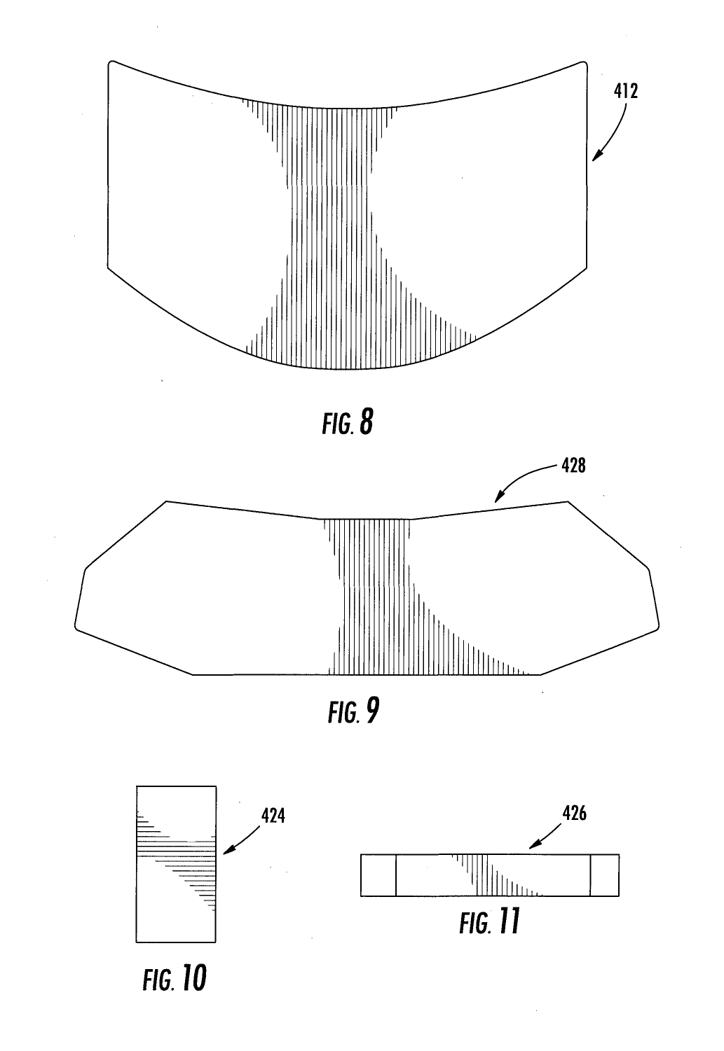

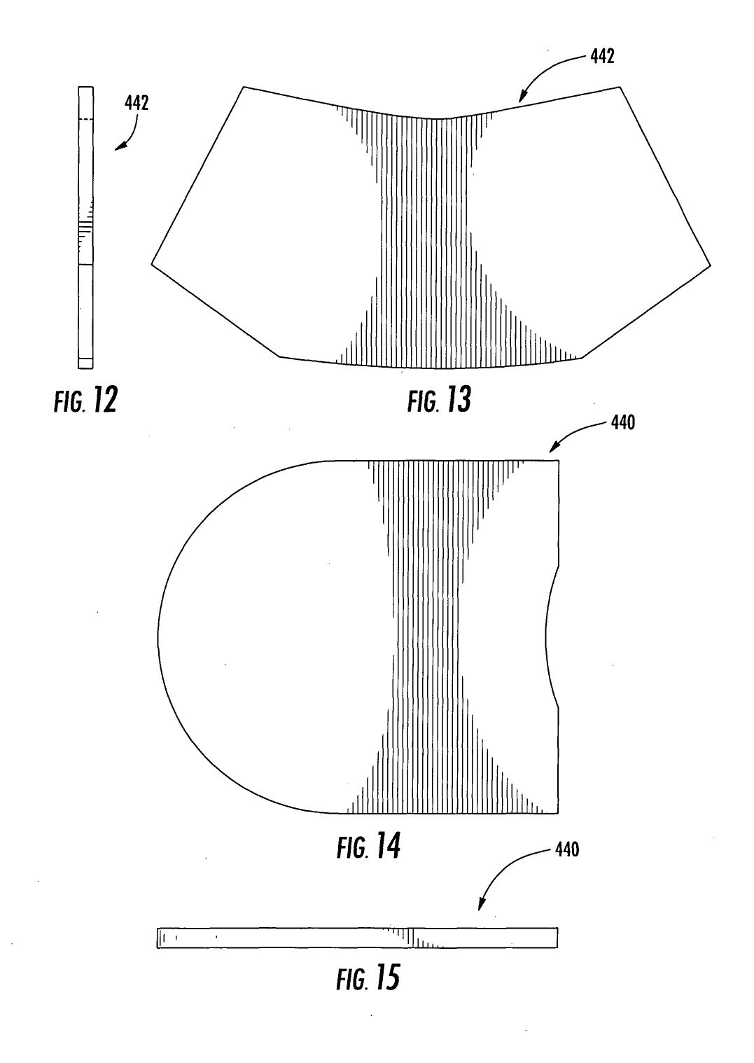

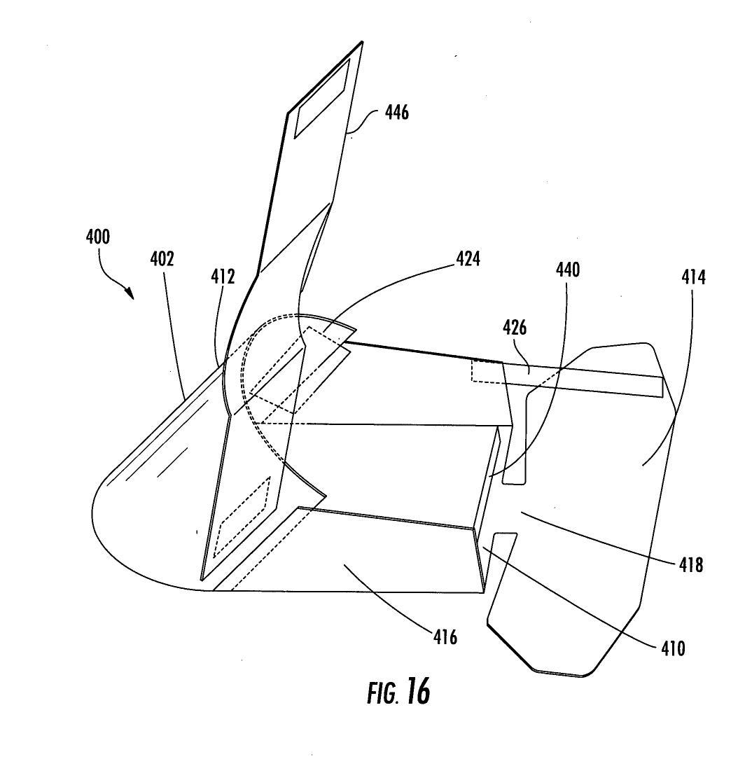

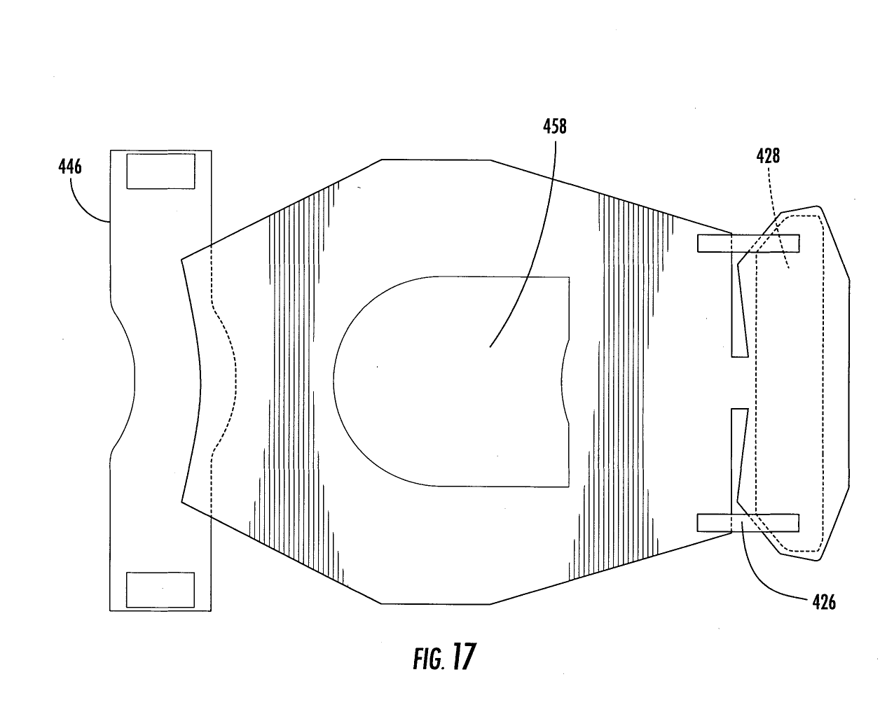

In some embodiments, as shown in FIGS. 5-15, the outer casing 402 includes a base portion 410 configured to be positioned under the foot or hoof, a front portion 412 configured to be positioned over the front of the foot or hoof, a back portion 414 configured to be positioned over the back of the foot or hoof, and side portions 416 configured to be positioned over the sides of the foot or hoof. In some embodiments, the outer casing 402 is formed of multiple components. For example, as shown in FIGS. 6 and 8, the base portion 410, the back portion 414, and the side portions 416 of the outer casing 402 may be formed as a single component, and the front portion 412 may be formed as a separate component. In some embodiments, the back portion 414 is connected to the base portion 410 by a flexible tether 418. In some embodiments, the front portion 412 is connected to each of the side portions 416 by an adhesive strip 424. In some embodiments, one of the side portions 416 is connected to an adhesive strip 426 configured to adhere to the back portion 414 and the other side portion 416 during application of the outer casing 402 onto a foot or hoof. The outer casing 402 may include an adhesive tail piece 428 for connecting the back portion 414 to the side portions 416. Specifically, the adhesive tail piece 428 may include an inner side including an adhesive for attaching to the back portion 414, and an outer side including an adhesive for attaching to the adhesive strip 426 and/or the side portions 416. In some embodiments, the outer casing 402 also includes a base stabilizing layer 440 positioned on and adhered to an interior surface of the base portion 410, and a front stabilizing layer 442 positioned on and adhered to an interior surface of the front portion 412.

Although the materials of construction of the outer casing 402 may vary, the outer casing 402 should be made out of highly durable materials that are able to withstand substantial forces and stresses without being damaged or compromising the integrity of the prefilled poultice wrap 200 received within the outer casing 402. For example, some or all of the base portion 410, the front portion 412, the back portion 414, and the side portions 416 of the outer casing 402 may be coated with a suitable high-performance plastic material. A non-limiting example of a suitable plastic material is a polyolefin film or similar flexible and high-performance film. Additionally, some or all of the base portion 410, the front portion 412, the back portion 414, and the side portions 416 of the outer casing 402 may be corona coated to permit printing on these portions. Accordingly, a logo or other visual markers may be printed on the outer casing 402 for advertising, branding or aesthetic purposes or for functional guidance in using the outer casing 402.

As discussed above, the prefilled poultice wrap 200 is configured to wrap around and contour to the foot or hoof of a human or animal. For example, the prefilled poultice wrap 200 may have an irregular elongated shape, substantially as shown in FIG. 3. Additionally, the pockets 204 may have certain irregular or regular shapes. In some embodiments, the prefilled poultice wrap 200 includes a base portion 222 and a front portion 224. The base portion may be configured to be positioned under the foot or the hoof, and the front portion may be configured to be positioned over the front of the foot or the hoof. In some embodiments, the base portion is shaped to substantially match the profile of the bottom of the foot or hoof. In this manner, the prefilled poultice wrap 200 may be configured to be positioned over and cover the entire foot or hoof, including the coronary band of a horse hoof. Accordingly, the prefilled poultice wrap 200 provides a specialized device for applying a poultice, as the prefilled poultice wrap 200 is shaped to target critical areas of the foot or hoof. Furthermore, the prefilled poultice wrap 200 and the pockets 204 may be formed in many different sizes for optimal use in various human and animal applications where the shapes and sizes of feet and hooves may vary widely. In some embodiments, the base portion 222 of the prefilled poultice wrap 200 is positioned against the base stabilizing layer 440 of the outer casing 402, and the front portion 224 of the prefilled poultice wrap 200 is positioned against the front stabilizing layer 442 of the outer casing 442. Accordingly, the prefilled poultice wrap 200 is held directly against the foot or hoof by the outer casing 402. The base stabilizing layer 440 and the front stabilizing layer 442 provide a non-slip barrier between the prefilled poultice wrap 200 and the base portion 410 and front portion 412 of the outer casing 402. Furthermore, the base stabilizing layer 440 and the front stabilizing layer 442 provide protection for sensitive or key problem areas of the foot or hoof, such as the coronary band of a horse hoof. In some embodiments, the prefilled poultice wrap 200 is removably fastened or adhered to the outer casing 402, so that the position of the prefilled poultice wrap 200 within the outer casing 402 will be maintained during use. For example, the prefilled poultice wrap 200 may be secured to the outer casing 402 with a temporary adhesive or a releasable fastener. In some embodiments, the base stabilizing layer 440 and the front stabilizing layer 442 are formed from a rubberized material. Although the prefilled poultice wrap system 300 has been described with reference to the prefilled poultice wrap 200, the system alternatively may include the prefilled poultice wrap 100 in conjunction with an outer casing 402 that is configured to receive the prefilled poultice wrap 100 and conform to a certain portion of the body.

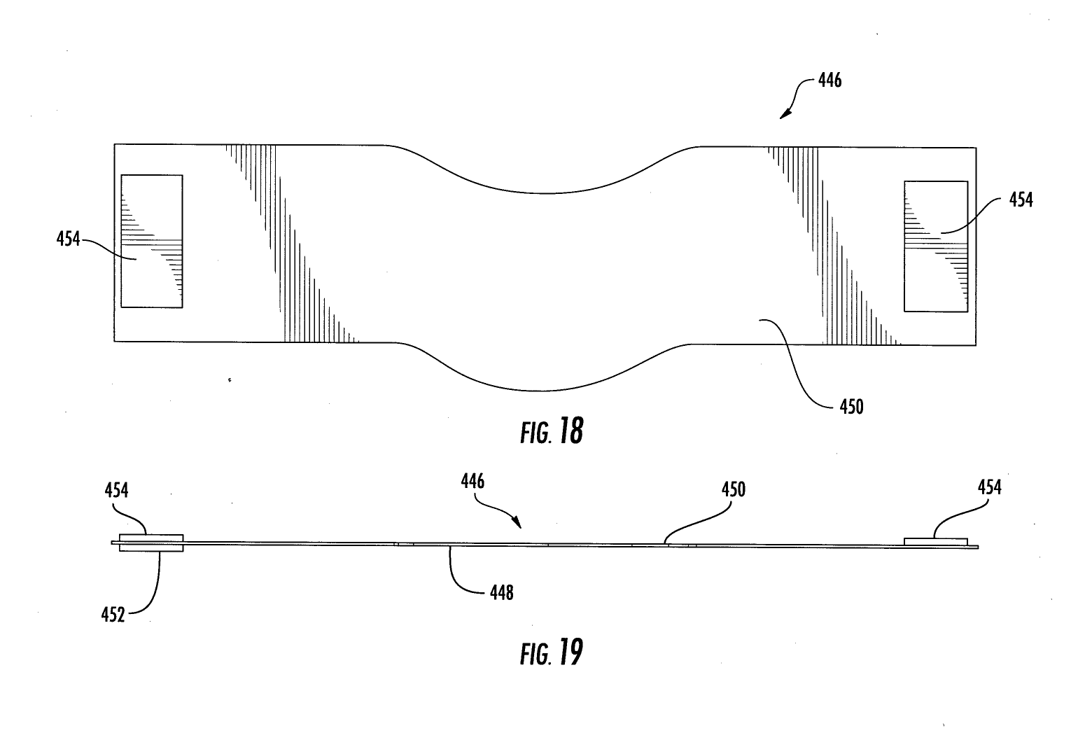

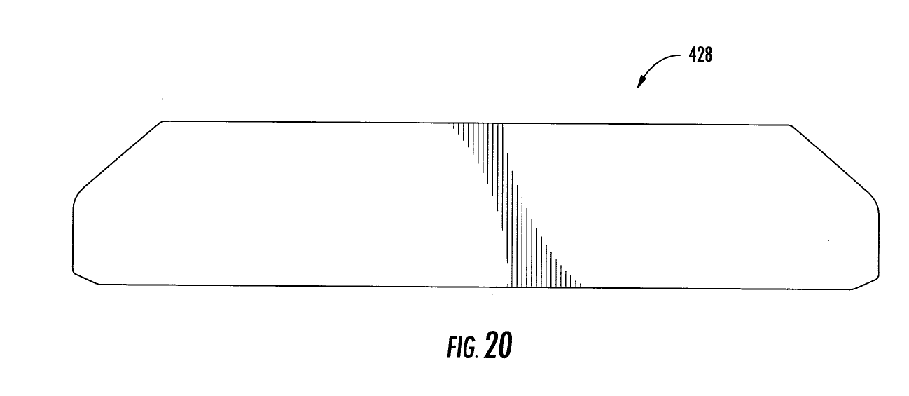

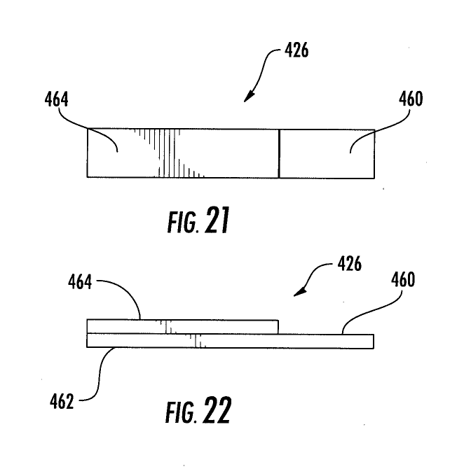

FIGS. 16-22 show an alternative embodiment of the outer casing 402, which similarly may be used in the prefilled poultice wrap system 300 to hold the prefilled poultice wrap 200 against the body of a human or animal. The embodiment of the outer casing 402 of FIGS. 16-22 generally may be configured in a manner similar to the embodiment of FIGS. 5-15 but may include certain structural and functional differences described herein below. As shown, the alternative embodiment of the outer casing 402 includes a base portion 410 configured to be positioned under the foot or hoof, a front portion 412 configured to be positioned over the front of the foot or hoof, a back portion 414 configured to be positioned over the back of the foot or hoof, and side portions 416 configured to be positioned over the sides of the foot or hoof. In some embodiments, the outer casing 402 is formed of multiple components. For example, as shown in the figures, the base portion 410, the back portion 414, and the side portions 416 of the outer casing 402 may be formed as a single component, and the front portion 412 may be formed as a separate component. In some embodiments, the back portion 414 is connected to the base portion 410 by a flexible tether 418. In some embodiments, the front portion 412 is connected to each of the side portions 416 by an adhesive strip 424. In some embodiments, one of the side portions 416 is connected to a hook and loop fastening back tie 426 configured to connect the one of the side portions 416 to the back portion 414 and/or the other side portion 416 during application of the outer casing 402 onto a foot or hoof. In some embodiments, the outer casing 402 also includes a base stabilizing layer 440 positioned on and adhered to an interior surface of the base portion 410.

Although the materials of construction of the outer casing 402 may vary, the outer casing 402 should be made out of highly durable materials that are able to withstand substantial forces and stresses without being damaged or compromising the integrity of the prefilled poultice wrap 200 received within the outer casing 402. For example, some or all of the base portion 410, the front portion 412, the back portion 414, and the side portions 416 of the outer casing 402 may be coated with a suitable high-performance plastic material. A non-limiting example of a suitable plastic material is a polyolefin film or similar flexible and high-performance film. Additionally, some or all of the base portion 410, the front portion 412, the back portion 414, and the side portions 416 of the outer casing 402 may be corona coated to permit printing on these portions. Accordingly, a logo or other visual markers may be printed on the outer casing 402 for advertising, branding or aesthetic purposes or for functional guidance in using the outer casing 402.

As discussed above, the outer casing 402 may be used in the prefilled poultice wrap system 300 to hold the prefilled poultice wrap 200 against the body of a human or animal. The prefilled poultice wrap 200 is configured to wrap around and contour to the foot or hoof of a human or animal. For example, the prefilled poultice wrap 200 may have an irregular elongated shape, substantially as shown in FIG. 3. Additionally, the pockets 204 may have certain irregular or regular shapes. In some embodiments, the prefilled poultice wrap 200 includes a base portion 222 and a front portion 224. The base portion 222 may be configured to be positioned under the foot or the hoof, and the front portion 224 may be configured to be positioned over the front of the foot or the hoof. In some embodiments, the base portion 222 of the prefilled poultice wrap 200 is positioned against the base stabilizing layer 440 of the outer casing 402, and the front portion 224 of the prefilled poultice wrap 200 is positioned against the front portion 412 of the outer casing 402. Accordingly, the prefilled poultice wrap 200 is held directly against the foot or hoof by the outer casing 402. The base stabilizing layer 440 provides a non-slip barrier between the prefilled poultice wrap 200 and the base portion 410 and front portion 412 of the outer casing 402. Furthermore, the base stabilizing layer 440 provides protection for sensitive or key problem areas of the foot or hoof. In some embodiments, the prefilled poultice wrap 200 is removably fastened or adhered to the outer casing 402, so that the position of the prefilled poultice wrap 200 within the outer casing 402 will be maintained during use. For example, the prefilled poultice wrap 200 may be secured to the outer casing 402 with a temporary adhesive or a releasable fastener. In some embodiments, the base stabilizing layer 440 is formed from a rubberized material. Although the prefilled poultice wrap system 300 has been described with reference to the prefilled poultice wrap 200, the system alternatively may include the prefilled poultice wrap 100, and the outer casing 402 may be configured to receive the prefilled poultice wrap 100 and conform to a certain portion of the body.

As noted above and shown in FIG. 16, the alternative embodiment of the outer casing 402 includes the hook and loop fastening back tie 426 instead of an adhesive strip for connecting one of the side portions 416 to the back portion 414 and/or the other side portion 416 during application of the outer casing 402. As shown, the hook and loop fastening back tie 426 may include an exposed adhesive surface 460 for attaching to the one of the side portions 416. Further, the back tie 426 may include a first side 464 including loop structure and a second side 462 including hook structure, wherein the second side 462 is longer than the first side 464. The back portion 414 of the outer casing 402 may include a tail piece 428 for connecting to the side portions 416. Specifically, the tail piece 428 may include an inner side including an adhesive for attaching to the back portion 414, and an outer side including loop structure for removably attaching to the second side of the back tie. In this manner, the back tie 426 may be used to removably connect the one of the side portions 416 to the back portion 414 and/or the other side portion 416 during application of the outer casing 402. As compared to the use of an adhesive strip, the back tie 426 may provide greater ease of use and versatility in adjusting the outer casing 402 around a portion of the body of a human or animal.

As shown in FIG. 16, the alternative embodiment of the outer casing 402 also includes a debris shield 446 configured to wrap around upper ends of the front portion 412, back portion 414, and side portions 416. In this manner, the debris shield 446 may closely contact the body of the human or animal along the upper ends of these portions to prevent debris from entering the outer casing 402. The debris shield 446 may include an inner side 448 having an adhesive for connecting the debris shield 446 to the upper end of the front portion 412. The inner side 448 of the debris shield 446 also may include an inner strip 452 having a hook structure and adhered near one end of the debris shield 446. The debris shield 446 also may include an outer side 450 having two outer strips 454, one of the outer strips 454 adhered near each end of the debris shield 446. Each of the outer strips 454 may have a loop structure. In this manner, the inner strip 452 and outer strips 454 allow the debris shield 446 to be removably secured around upper ends of the front portion 412, back portion 414, and side portions 416. Specifically, the inner strip 452 and outer strips 454 of the debris shield 446 may be attached to one another, to the back tie 426, to the tail piece 428, or to additional portions of the outer casing 402. In this manner, the inner strip 452 and outer strips 454 of the debris shield 446 may provide ease of use and versatility in securing the outer casing 402 around a portion of the body of a human or animal to prevent debris from entering the outer casing 402.

The alternative embodiment of the outer casing 402 further includes a bottom reinforcement insert 458 positioned on the base portion 410 of the outer casing 402. In this manner, the bottom reinforcement insert 458 may provide improved structural strength and wear resistance, particularly for applications where the outer casing 402 is configured to wrap around and contour to the foot or hoof of a human or animal and thus the base portion 410 is subjected to substantial forces and wear.

In another aspect, the present disclosure includes a method of applying a prefilled wrap to the body of a human or animal. The method includes the step of providing a prefilled wrap including a matrix defining a plurality of pockets therein. The prefilled wrap also includes at least one dry ingredient formulation contained in each of the plurality of pockets. The method also includes the step of reconstituting the prefilled wrap by applying a reconstituting fluid to the at least one dry ingredient formulation. The method further includes the step of wrapping the reconstituted poultice wrap around a portion of the body of the human or animal.

According to one embodiment, the present disclosure provides a method of applying a prefilled poultice wrap 100 to the body of a human or animal. The method includes the step of providing a prefilled poultice wrap 100 including a matrix 102 defining a plurality of pockets 104 therein. The prefilled poultice wrap 100 also includes a dry poultice formulation 106 contained in each of the plurality of pockets 104. The method also includes the step of reconstituting the prefilled poultice wrap 100 by applying a reconstituting fluid to the at least one dry poultice formulation 106. The method further includes the step of wrapping the reconstituted poultice wrap 100 around a portion of the body of the human or animal. In some embodiments, the step of reconstituting the prefilled poultice wrap 100 includes reconstituting the dry poultice formulation 106. In some embodiments, the step of reconstituting the prefilled poultice wrap 100 includes soaking the prefilled poultice wrap 100 in water for approximately 15 to 20 seconds. In this manner, the dry poultice formulation 106 may absorb its maximum capacity of water without excessively hydrating or diluting the dry poultice formulation 106 and without needing to wring out the prettied poultice wrap 100 prior to wrapping it around a portion of the body. In some embodiments, the dry poultice formulation 106 is configured to absorb more water than the matrix 102. In some embodiments, the prefilled poultice wrap 100 is soaked in cold water, such as ice water, to provide the dual-action effect described above. In other embodiments, the prefilled poultice wrap 100 is soaked in warm water to provide a warming effect. In some embodiments, upon reconstituting the prefilled poultice wrap 100, the reconstituted poultice formulation 106 assumes a gel-like consistency. In some embodiments, the step of wrapping the reconstituted poultice wrap 100 around a portion of the body of the human or animal includes molding the reconstituted poultice wrap 100 around the portion of the body. In some embodiments, the gel-like consistency of the reconstituted poultice formulation 106 allows the reconstituted poultice wrap 100 to be molded around the portion of the body.

In yet another aspect, the present disclosure includes a method of applying a prefilled wrap system to the body of a human or animal. The method includes the step of providing a prefilled wrap system including a protective outer casing and a prefilled wrap received in the outer casing. The prefilled wrap includes a matrix defining a plurality of pockets therein. The prefilled wrap also includes at least one dry ingredient formulation contained in each of the plurality of pockets. The method also includes the step of reconstituting the prefilled wrap by applying a reconstituting fluid to the at least one dry ingredient formulation. The method further includes the step of positioning the prefilled wrap system over a portion of the body of the human or animal, so that the outer casing holds the prefilled wrap against the portion of the body.

According to one embodiment, the present disclosure provides a method of applying a prefilled poultice wrap system 300 to the body of a human or animal. The method includes the step of providing a prefilled poultice wrap system 300 including an outer casing 402 and a prefilled poultice wrap 200 received in the outer casing 402. The prefilled poultice wrap 200 includes a matrix 202 defining a plurality of pockets 204 therein. The prefilled poultice wrap 200 also includes a dry poultice formulation 206 contained in each of the plurality of pockets 204. The method also includes the step of reconstituting the prefilled poultice wrap 200. The method further includes the step of positioning the prefilled poultice wrap system 300 over a portion of the body of the human or animal, so that the outer casing 402 holds the prefilled poultice wrap 200 against the portion of the body. In some embodiments, the method further comprises securing the prefilled poultice wrap system 300 over the portion of the body. In some embodiments, the outer casing 402 is configured to wrap around and contour to a foot or hoof of the human or animal, and the prefilled poultice wrap 200 is configured to wrap around and contour to the foot or hoof. In some embodiments, the outer casing 402 and the prefilled poultice wrap 200 are configured to fit feet or hooves of all sizes. In some embodiments, the outer casing 402 may be another type of covering device configured to wrap around and contour to a different portion of the human or animal, such as an arm, leg, elbow, or knee. In some embodiments, the entire prefilled poultice wrap system 300 is disposable. In other embodiments, the prefilled poultice wrap 200 is disposable and the outer casing 402 is reusable.

In still another aspect, the present disclosure includes a method of manufacturing a prefilled wrap to apply to the body of a human or animal. The method includes the step of providing a first sheet, a second sheet, and at least one dry ingredient formulation. The method also includes the step of depositing the at least one dry ingredient formulation between the first sheet and the second sheet. The method further includes the step of sealing the first sheet to the second sheet to form a prefilled wrap including a matrix defining a plurality of pockets between the first sheet and the second sheet, so that the at least one dry ingredient formulation is contained in each of the plurality of pockets.

According to one embodiment, the present disclosure provides a method of manufacturing a prefilled poultice wrap 100 to apply to the body of a human or animal. The method includes the step of providing a first sheet 110, a second sheet 112, and at least one dry poultice formulation 106. The method also includes the step of depositing the at least one dry poultice formulation 106 between the first sheet 110 and the second sheet 112. The method further includes the step of sealing the first sheet 110 to the second sheet 112 to form a prefilled poultice wrap 100 including a matrix 102 defining a plurality of pockets 104 between the first sheet 110 and the second sheet 112, so that the at least one dry poultice formulation 106 is contained in each of the plurality of pockets 104. In some embodiments, the first sheet 110, the second sheet 112, and the at least one dry poultice formulation 106 are provided in a dry state. In some embodiments, the method further includes packaging the prefilled poultice wrap 100 in a dry state for future use. In some embodiments, all steps of the method are carried out in a dry state. Accordingly, the method does not include soaking or dipping the matrix in a fluid ingredient batch of poultice material and subsequently drying. Additionally, the method does not include spraying or impregnating the matrix with a fluid ingredient spray of poultice material and subsequently drying. The specific composition of the dry poultice formulation 106 may be varied for different applications of the prefilled poultice wrap 100 without substantially altering the structure of the prefilled poultice wrap or the method of manufacturing the prefilled poultice wrap.

Although specific embodiments of the disclosure have been described, one of ordinary skill in the art will recognize that numerous other modifications and alternative embodiments are within the scope of the disclosure. For example, while embodiments of the disclosure have been described with respect to specific configurations and relationships, it will be appreciated that numerous configurations and relationships are within the scope of this disclosure. Furthermore, although embodiments have been described in language specific to structural elements and features, it will be understood that the disclosure is not necessarily limited to the specific elements or features described. Rather, the specific elements and features are disclosed as illustrative forms of implementing the embodiments.

Conditional language, such as, among others, “can,” “could,” “might,” or “may,” unless specifically stated otherwise, or otherwise understood within the context as used, is generally intended to convey that certain embodiments could include, while other embodiments do not include, certain elements or features. Thus, such conditional language is not generally intended to imply that elements or features are in any way required for one or more embodiments.

We claim:

1. A prefilled poultice wrap for applying to the body of a human or animal, the prefilled poultice wrap comprising:
a matrix defining a plurality of pockets therein; and
a dry poultice formulation contained in each of the plurality of pockets.

2. The prefilled poultice wrap of claim 1, wherein the matrix comprises a first sheet and a second sheet sealed to one another to define the plurality of pockets between the first sheet and the second sheet.

3. The prefilled poultice wrap of claim 2, wherein the first sheet and the second sheet are sealed to one another by a heat/pressure combination seal.

4. The prefilled poultice wrap of claim 2, wherein the first sheet and the second sheet are formed of a fluid-permeable material configured to allow a reconstituting fluid to enter the plurality of pockets to reconstitute the dry poultice formulation.

5. The prefilled poultice wrap of claim 4, wherein the first sheet and the second sheet are formed of a water-permeable material configured to allow water to enter the plurality of pockets to reconstitute the dry poultice formulation.

6. The prefilled poultice wrap of claim 5, wherein the first sheet and the second sheet are configured to substantially retain the reconstituted poultice formulation in each of the plurality of pockets.

7. The prefilled poultice wrap of claim 4, wherein the dry poultice formulation substantially fills each of the plurality of pockets when the prefilled poultice wrap is in a dry state, and wherein the reconstituted poultice formulation completely fills each of the plurality of pockets when the prefilled poultice wrap is in a reconstituted state.

8. The prefilled poultice wrap of claim 2, wherein the first sheet and the second sheet are formed of a filter paper.

9. The prefilled poultice wrap of claim 2, wherein the dry poultice formulation comprises guar gum.

10. The prefilled poultice wrap of claim 9, wherein the dry poultice formulation further comprises Epsom salt.

11. The prefilled poultice wrap of claim 10, wherein the dry poultice formulation further comprises clay.

12. The prefilled poultice wrap of claim 1, wherein the matrix has an irregular shape configured to substantially match a portion of the body of the human or animal.

13. The prefilled poultice wrap of claim 1, wherein the matrix is configured to wrap around and contour to a leg or arm of the human or animal.

14. The prefilled poultice wrap of claim 1, wherein the matrix is configured to wrap around and contour to a foot or hoof of the human or animal.

15. The prefilled poultice wrap of claim 14, wherein the matrix comprises a base portion configured to be positioned under the foot or hoof, and wherein the matrix comprises a front portion configured to be positioned over the front of the foot or hoof.

16. The prefilled poultice wrap of claim 15, wherein the base portion is shaped to match the profile of the bottom of the foot or hoof.

17. The prefilled poultice wrap of claim 16, wherein at least one of the plurality of pockets is defined in the base portion of the matrix, and wherein at least one of the plurality of pockets is defined in the front portion of the matrix.

18. A prefilled poultice wrap system for applying to the body of a human or animal, the system comprising:
a case configured to wrap around a portion of the body of the human or animal; and
a prefilled poultice wrap removably received in the case;
wherein the prefilled poultice wrap comprises:
a matrix defining a plurality of pockets therein, and
a dry poultice formulation contained in each of the plurality of pockets; and
wherein the case is configured to hold the prefilled poultice wrap against the body of the human or animal.

19. A method of applying a prefilled poultice wrap to the body of a human or animal, the method comprising:
providing a prefilled poultice wrap, wherein the prefilled poultice wrap comprises:
a matrix defining a plurality of pockets therein; and
at least one dry poultice formulation contained in each of the plurality of pockets;
reconstituting the prefilled poultice wrap by applying a reconstituting fluid to the at least one dry poultice formulation; and
wrapping the reconstituted prefilled poultice wrap around a portion of the body of the human or animal.

20. A method of manufacturing a prefilled poultice wrap for applying to the body of a human or animal, the method comprising:
providing a first sheet, a second sheet, and at least one dry poultice formulation;
depositing the at least one dry poultice formulation between the first sheet and the second sheet; and
sealing the first sheet to the second sheet to form a matrix defining a plurality of pockets between the first sheet and the second sheet, wherein the at least one dry poultice formulation is contained in each of the plurality of pockets.
